(19) United States
(12) Patent Application Publication
(10) Pub. No.: US 2002/0022231 A1
(43) Pub. Date: Feb. 21, 2002

(54) Method for identifying modulators of DNA structure-specific binding proteins by high-throughput screening

(76) Inventor: Vaughn Vasil, Alameda, CA (US)

Correspondence Address: VAUGHN SMIDER, 1823-A PEARL ST, ALAMEDA, CA, 94501, US

(21) Appl. No.: 09/931,883

(22) Filed: Aug. 20, 2001

Related U.S. Application Data
(60) Provisional application No. 60/226,441, filed on Aug. 18, 2000.

(51) Int. Cl. C12Q 1/68

(52) U.S. Cl. 435/6

(57) ABSTRACT

The invention relates to a solid phase DNA structure-specific binding protein (DSSBP) assay for identifying compounds which modulate DNA structure-specific binding proteins. The assay has a model DNA substrate adsorbed on a solid support, a DNA- structure-specific binding protein known to bind to the DNA substrate, and a mechanism to detect the DNA structure-specific binding protein. This assay allows rapid detection of pharmaceutically important DNA-binding-protein modulators.

### CROSS-REFERENCE TO RELATED APPLICATIONS

This application claims the priority benefit of the provisional patent application U.S. Ser. No. 60/226,441 filed Aug. 18, 2000, which is incorporated by reference in its entirety.

### FIELD OF THE INVENTION

This invention relates to a solid phase assay to measure the ability of a test substance to modulate DNA structure-specific binding proteins (DSSBPs). This assay advantageously simplifies quantitation of DSSBP binding activity, and is especially well suited for rapid analysis of large numbers of individual samples. The assays and methods of this invention may be used to identify pharmaceutically important DSSBP modulators.

### BACKGROUND OF THE INVENTION

DNA structure-specific binding proteins (DSSBPs) are a large class of proteins capable of contacting DNA in cells. DSSBPs may participate in chromosome structure, DNA replication, RNA transcription, DNA repair, or DNA recombination. Many of these proteins have already been proven to have significant pharmaceutical relevance. DNA binding proteins involved in replication are targeted by several drugs: 1) etoposide, a chemotherapeutic agent, binds to topoisomerases which are enzymes that function during DNA replication; 2) HIV reverse transcriptase is modulated by several drugs including acyclovir; 3) helicases in bacteria are inhibited by nalidixic acid and ciprofloxacin.

Several DNA structures and their binding proteins are important in cellular physiology, and could be therapeutic targets. Damaged DNA includes many DNA structures which are substrates for DNA repair enzymes. Such structures include DNA ends, abasic sites, pyrimidine dimers, DNA nicks, or DNA mismatches [Friedberg et al., DNA Repair and Mutagenesis (1995)]. Additionally, there are several pharmaceutically relevant proteins which act through binding DNA. Examples of these include p53, which is a DNA binding protein that is mutated in many human cancers, and DNA-PK which is a DNA repair protein that binds to DNA ends.

Methods to rapidly detect the DNA binding activity of DSSBPs are not currently available. Previous methods have relied on electrophoretic mobility shift assays which require the time consuming and labor intensive task of pouring and running acrylamide gels, or filter binding assays which do not allow rapid and simultaneous analyses of large numbers of samples [Hwang et al., Methods in Molecular Biology: DNA Repair Protocols 113: 103-20 (1999); Montiminy & Peers, U.S. Pat. No. 5,849,493 (1998)]. Assays utilizing the solid phase have been reported for helicase enzymes [Crute, U.S. Pat. No. 5,958,696 (1999); Giordano et al., U.S. Pat. No. 5,705,344 (1998)], and for transcription factors [Peterson et al., U.S. Pat. No. 5,563,036 (1996)]. These methods, however, do not provide the means to detect the binding of structure-specific DNA binding proteins.

The recent advent of large and diverse chemical libraries for the purposes of drug discovery, the improvements in robotics and microfluidics applied to bioinstrumentation, and the rapidly expanding array of bioinformation makes the development of high-throughput screening assays of paramount importance to the pharmaceutical industry. The present invention allows such high-throughput screening for drug targets in the DNA structure-specific binding protein category.

### SUMMARY OF THE INVENTION

This invention overcomes the drawbacks associated with the previous DSSBP assays by providing a solid phase assay in which the presence of the DNA binding protein bound to DNA may be readily and quantitatively detected.

The prime objective of the invention is to provide a solid phase assay comprising a model DSSBP DNA substrate bound to a solid support, wherein the model substrate comprises single or double stranded nucleic acid. A DSSBP is allowed to bind to the substrate in the presence or absence of a test substance, and detection may be accomplished by several mechanisms. Potential means to detect DSSBP activity include: 1) direct detection through an attached label on the DSSBP; 2) detection of a function of the DSSBP, like a kinase activity or other biochemical activity such as ligand binding, protease activity, phosphatase activity, 3) through addition of a detector molecule which binds to the DSSBP, wherein the detector molecule comprises a molecule with an attached label, or 4) addition of a detector molecule which binds to the DSSBP, followed by addition of a secondary detector molecule which binds the first detector molecule, wherein the secondary detector molecule comprises a molecule with an attached label. Often primary and secondary molecules are antibody molecules. The attached label in any of these possibilities may be a fluorescent molecule, a dye producing a color change, a radioactive atom, an enzyme capable of catalyzing a reaction producing a detectible reaction product, etc. These detection methods are well known to those skilled in the art.

One objective of the present invention is to provide a method for measuring the DNA-structure specific binding activity of a test protein comprising the steps of:

a) Contacting an immobilized nucleic acid substrate containing a specific structure with a test protein or mixture of proteins.

b) detecting the protein or mixture of proteins from step (a) bound to the immobilized nucleic acid substrate.

Another objective is to provide a method for measuring the DNA-binding-protein modulating ability of a test substance comprising the steps of:

a) Contacting an immobilized nucleic acid substrate containing a specific structure with a test substance to produce a reaction premix,

b) Contacting the reaction premix of step (a) with a DSSBP capable of binding the immobilized substrate to produce a reaction mix,

c) detecting the DSSBP of step (b) bound to the immobilized nucleic acid substrate.

### BRIEF DESCRIPTION OF THE DRAWINGS

FIG. 1. Schematic drawing of the components of the assay. These components include 1) a solid support, 2) a DNA substrate, 3) a DSSBP capable of binding said DNA substrate, and 4) a detection method. In this figure, the solid support is a streptavidin coated 96-well plate, the DNA substrate is a biotinylated linear DNA molecule containing a DNA end, the DSSBP is the DNA-PK protein complex, and the detection method is an anti-DNA-PK antibody bound by a secondary antibody conjugated to the horseradish peroxidase enzyme which catalyzes a fluorogenic reaction. This assay could also be applied wherin other DNA structures are present, and would be bound by other structure-specific DNA binding proteins.

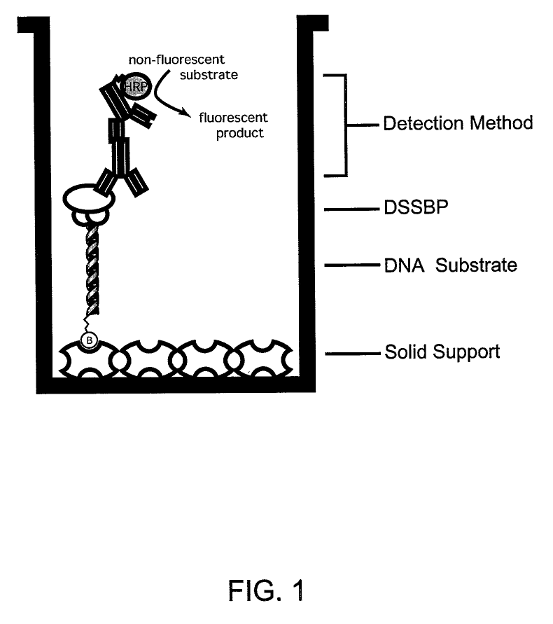

FIG. 2. Proof of concept bar graph, showing an increased fluorescence signal in lane 7, which contains all components of assay. If any of the components of the assay are ommitted (lanes 1-6) signal is severely diminished. Labels are Ab=anti-DNA-PK antibody, XT=HeLa nuclear extract, fBS=linear biotinylated Bluescript DNA.

FIG. 3. Bar graph showing that biological activity of the extract is required to produce a signal in the assay. Heated extract shows diminished activity compared to unheated extract (compare lanes 3 and 4).

FIG. 4. The assay specifically detects DNA-PK bound to DNA ends. Anti-ATM antibodies do not produce signal compared to anti-DNA-PK antibodies (compare lane 3 to lane 5).

FIG. 5. Purified DNA-PK produces signal comparable to HeLa nuclear extracts. Significant binding is seen with pure DNA-PK (lane 2), or HeLa nuclear extract (lane 4), but not the single-stranded binding protein (SSB, lane 3).

### DETAILED DESCRIPTION OF THE INVENTION

The present invention provides a method to screen for compounds that modulate DNA structure specific binding proteins (DSSBPs). Identification of such compounds has significance in the pharmaceutical and medical industries. Compounds that modulate DSSBPs are potential drugs for several diseases, including cancer. Modulation of DSSBPs in cancer could enhance radiation therapy or chemotherapy. Additionally, certain viruses, like HIV, utilize DSSBPs in their lifecycle. Thus, compounds that modulate DSSBPs could be developed into important antiviral drugs.

Also, the ability to detect DSSBPs reliably and quickly could be useful in certain diagnostic procedures, such as to detect when a DSSBP is present in high or low concentration as a result of a particular therapy, or prior to instigation of a therapy.

Methods to detect DSSBPs in a high-throughput fashion have not been available until the present invention.

For the purposes of describing this invention the following terms will be helpful and will have the following meanings:

### Definitions

The term “DNA” refers to deoxyribonucleic acid. It will be understood by those of skill in the art that where manipulations are described herein that relate to DNA they will also apply to RNA.

The term “specific structure” or “specific DNA structure” as used herein refers to any DNA structure not present in supercoiled plasmid DNA. DNA ends, nicks, covalently attached moieties, mismatched bases, chemical changes induced by ultraviolet or gamma irradiation, and protein/DNA complexes such as chromatin are all considered specific DNA structures as used herein.

The term “DNA ends” or ends refers to the position in a DNA strand wherein a phosphodiester bond is broken. In a single-stranded DNA end a nucleotide is only covalently linked with one other nucleotide. A “double-stranded DNA or RNA end” refers to the position in a double-stranded DNA molecule wherein the molecule is no longer double-stranded. Generally DNA ends are recognizable to those skilled in the art. Double-stranded DNA ends are characterized as blunt, having a 5′ overhang, a 3′ overhang, or a hairpin structure. A DNA end may or may not contain a 5′ phosphate group.

As used herein “DNA damage” or “damaged DNA” refers to DNA that is not normally present in an intact cell under physiological conditions during interphase. DNA ends, for instance, are not produced normally in an interphase cell, except for the special case of telomeres which contain DNA ends. DNA ends are produced, however, by certain drugs or irradiation like gamma rays or ultraviolet rays. For the purposes of this invention DNA ends are considered damaged DNA. Other DNA damage may include the covalent attachment of chemical moieties to DNA, such that the DNA no longer comprises the natural structure it has in an intact interphase cell.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

The term “mutations” means changes in the sequence of a wild-type nucleic acid sequence or changes in the sequence of a peptide. Such mutations may be point mutations such as transitions or transversions. The mutations may be deletions, insertions or duplications.

As used herein the term “physiological conditions” refers to temperature, pH, ionic strength, viscosity, and like biochemical parameters which are compatible with a viable organism, and/or which typically exist intracellularly in a viable cultured yeast cell or mammalian cell. For example, the intracellular conditions in a yeast cell grown under typical laboratory culture conditions are physiological conditions. Suitable in vitro reaction conditions for in vitro transcription cocktails are generally physiological conditions. In general, in vitro physiological conditions comprise 50-200 mM NaCl or KCl, pH 6.5-8.5, 20-45° C. and 0.001-10 mM divalent cation (e.g., Mg++, Ca++); preferably about 150 mM NaCl or KCl, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions may be selected by the practitioner according to conventional methods. For general guidance, the following buffered aqueous conditions may be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants.

As used herein the term “solid support” refers to any material or compound which will not dissolve in a solution to which it is in contact. The solution may be organic or inorganic, aqueous or inaqueous. Thus, a solution is easily separable from a solid support by means well known to those in the art. For example a solid support may be the surface of a vessel, whereby the solution is removed easily from the solid support by removing the solution from the vessel. Alternatively, a solid support may be a substance that is easily removed from the solution by centifugation. Also, a solid support may be a material, or a portion thereof, that is metallic or magnetic such that the solid support and a solution may be separated by the use of a magnet.

The term “immobilized” means that a molecule or material is bound either covalently or noncovalently to a solid support. Hence, an “immobilized nucleic acid” means that a nucleic acid is attached to a solid support.

The term “microtiter plate” as used herein refers to the physical linkage of at least two vessels. More often a microtiter plate comprises the physical linkage of several vessels, such as 96 vessels in an array format. A microtiter plate may have less than, or more than 96 wells.

### Solid-support binding assay

Solid support materials that can be used for this assay include any conventional support materials, including (but not limited to) polystyrene, polyvinyl chloride or polycarbonate microtiter plates or beads and derivatized argarose or acrylamide beads. Often, for high throughput assays, the support materials are comprised of microtiter plates. The surface of the solid support material can be derivatized with a protein. However, other small DNA-interaction promoting materials (e.g., glycine) may also be used. Solid support materials comprising certain esters can be reacted with amine containing second molecules, such as a nucleic acid derivatized with an amino group at either the 5′ or 3′ end, to produce a solid support comprising a covalently coupled second molecule. Often solid supports derivatized with a protein can be further derivatized with another molecule. For instance, solid supports derivatized with the protein streptavidin can be further derivatzed by contacting the solid support with a molecule containing biotin. Biotin binds strongly to streptavidin, so any molecule comprising a biotin group will be bound to the solid support. Polystyrene microtiter plates derivatized with a protein like streptavidin are commercially available (Pierce, Rockford, Ill.), or may be prepared by adsorbing the protein of interest with underivatized, high protein binding plates. In one embodiment, polystyrene plates derivatized with streptavidin are utilized as the solid support.

A polynucleotide with the relevant structure may be prepared by several methods. If the DSSBP is an end-binding protein, DNA may be prepared by digesting a plasmid DNA molecule with a restriction enzyme to generate an end. If the DSSBP of interest binds UV damaged DNA, the DNA of interest may be irradiated with UV light. If the DSSBP binds DNA nicks, nicked DNA may be produced by incubation of a DNA molecule with an endonuclease such as DNase I, or irradiated with gamma radiation. If the DSSBP binds a chromatin structure, these can be produced by allowing histones to interact with a representative DNA molecule to produce the relevant nucleosome structure of interest. Telomeric structures can be synthesized which contain telomere-specific nucleotide residues in the context of a DNA end. Other DNA structures may be synthesized by chemical means well known to those skilled in the art. All of the aforementioned DNA structures can then be adsorbed to the solid support by several different mechanisms.

DNA may be biotinylated by several means. Plasmid DNA can be restricted by an endonuclease which leaves a 5′ overhang and serves as a substrate for DNA polymerase. A biotinylated nucleotide, like biotin-16-dUTP (10 mM) can be included in a reaction mix consisting of dCTP, dGTP, and dATP (at 1 mM each), magnesium chloride (10 mM), sodium chloride (100 mM), buffer, and Klenow polymerase (1U). The polymerase incorporates the biotinylated nucleotide into the DNA strand, which will allow it to be adsorbed to the solid support. Alternatively, a biotinylated oligonucleotide can be synthesized and used in the polymerase chain reaction (PCR) to produce the biotinylated DNA of interest. The resulting biotinylated DNA can be purified by spun column chromatography, or by other means known in the art such as HPLC or gel purification.

Adsorption of the biotinylated DNA to the streptavidin coated plates can be accomplished by incubating 0.01 g biotinylated DNA to 100 μg biotinylated DNA in a standard buffer such as 10 mM Tris, 5 mM EDTA for 20 minutes at room temperature. Preferably, the amount of DNA is between 0.1 and 10 μg. Unbound DNA can then removed, and the wells are washed with a standard buffer like TEN buffer (10 mM Tris, 1 mM EDTA, 100 mM NaCl, pH 7.4).

Following adsorption of the DNA to the solid support, the preferred embodiment allows for a step whereby non-specific protein is allowed to adsorb to the solid support. This step is to minimize background signal. Several nonspecific proteins may be used including, but not limited to, bovine serum albumin or milk proteins. For example, up to 500 l of a solution comprising 5% non-fat dried milk, 10 mM Tris, 1 mM EDTA, 0.05% Tween-20 can be incubated with the solid support for between 30 min and 24 hours, preferably between 1 hour and 12 hours. This solution can then be removed and the solid support washed with a buffer like TEN.

In one embodiment, test substances are used to modulate DSSBP activity. A reaction premix can be formed by addition of a solution containing the test compound to the solid support. Test compounds may be small organic molecules, carbohydrates, lipids, or protein molecules, including antibodies, and may be added at any concentration of interest to the investigator.

The source of the DSSBP may vary greatly depending on the investigator's area of interest. Several DNA binding proteins are found in the nucleus, therefore nuclear extract is a good source for some DSSBPs. Nuclear extract may be prepared as described [Hwang et al., Methods in Molecular Biology: DNA Repair Protocols 113: 103-20 (1999)]. Other DSSBPs may be cytoplasmic, or may be produced by genetic engineering in prokaryotic or eukaryotic cells. Proteins purified by several mechanisms would also be a useful source of protein. In one embodiment, nuclear extract from HeLa cells is incubated with the solid support at a concentration of from 0.01 mg/ml to 10 mg/ml in a volume of 0.5 l to 5 ml for 1 minute to 30 minutes at from 0° C. to 50° C. This reaction may include a test compound formed in a premix as aforementioned. Following this binding reaction, the unbound protein is removed and the solid support can be washed with a buffer like TEN.

Once the binding reaction has occurred, detection of the DSSBP/DNA complex on the solid support can be accomplished by several means. The DSSBP itself may be labeled radioactively, fluorescently, calorimetrically, or conjugated to an enzyme capable of catalyzing a reaction that produces a detectible signal. Also, the DSSBP may be detected by detecting an activity of the DSSBP, like a kinase or other biochemical activity. Alternatively, the complex may be detected by incubating a molecule that is capable of binding the DSSBP with the DSSBP/DNA complex on the solid support. This protein can be an antibody specific to the DSSBP. This antibody may be labeled radioactively, fluorescently, calorimetrically, or conjugated to an enzyme capable of catalyzing a reaction that produces a detectible signal. Alternatively, a secondary antibody which binds the primary antibody may be incubated with the solid support containing the DSSBP/DNA/Ab complex. The technique of using a secondary antibody to detect proteins is well known to those skilled in the art. This secondary antibody may be labeled radioactively, fluorescently, calorimetrically, or conjugated to an enzyme capable of catalyzing a reaction that produces a detectible signal.

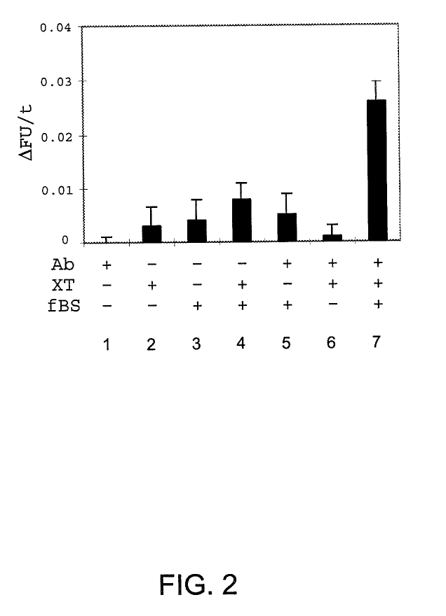

The solid phase DSSBP assay of this invention advantageously measures both accurately and quickly the extent to which a test substance inhibits DNA binding activity. For example, once the microtiter plates used for the assays were streptavidin-derivatized and DNA substrates adsorbed, the experiments depicted in FIGS. 2-5 were assembled, run, and processed in a period of only 2 hours each. As these experiments are capable of processing nearly 100 samples without significant increases in time, the improvement in efficiency is substantial compared to alternative methods.

### EXAMPLE

Several pharmacologically important DNA structure-specific binding proteins exist. The DNA-dependent protein kinase is known to be involved in DNA-double-strand break repair [Smider & Chu, Sem. Immun. 9:189-97 (1997)], and is essential to radiation resistance in mammalian cells [Danska et al., Mol. Cell. Biol. 16: 5507-17 (1996); Kirchgessner et al., Science 267:1178-85 (1995)]. This protein complex binds to the structure of a DNA end [Anderson, Trends Biochem. Sci. 18:433-7 (1993)]. Thus, DNA-PK is a useful and important example of a DSSBP capable of analysis using a solid phase structure-specific DNA binding assay.

The ability to detect the DNA-PK complex was examined under a variety of conditions (FIG. 2). Biotinylated fBS linear plasmid was either adsorbed (FIG. 2, lanes 3, 4, 5, 6, 7) to streptavidin-coated microtiter plates, or ommitted (FIG. 2, lanes 1, 2, 6). Plates were then blocked with blocking solution and HeLa nuclear extract was added (FIG. 2, lanes 2, 4, 6, 7), or ommitted (FIG. 2, lanes 1, 3, 5) in a 10 minute reaction. Wells were either washed with polyclonal anti-DNA-PK antibody (Serotec, Raleigh, N.C.) diluted 1:1000 in wash solution (FIG. 2, lanes 1, 5, 6, 7) or with wash solution alone (FIG. 2, lanes 2, 3, 4). After washing, the DNA-PK/antibody complex was detected with anti-Rabbit-immunoglobulin-HRP conjugate and the fluorescent substrate Quanta-Blu (Pierce, Rockford, Ill.) on an fMAX microtiter plate fluorometer (Molecular Devices, Sunnyvale, Calif.). Kinetic reads measured change in fluorescent units per time. Error bars represent standard errors for duplicate wells. Full fluorescence requires DNA, HeLa extract, and anti-DNA-PK antibodies.

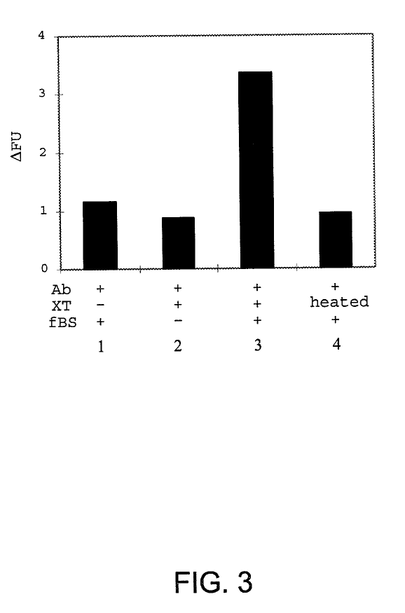

Biologically active DNA-PK is required to produce the signal shown in FIG. 2. Heat denatured nuclear extract is unable to produce the signal of untreated nuclear extract (FIG. 3, compare lanes 3 and 4). This is important since protein is readily adsorbed to polystyrene plates (such as in immunosorbant assays), and thus a signal independent of biological activity might be possible under suboptimal conditions of the assay—especially suboptimal blocking conditions.

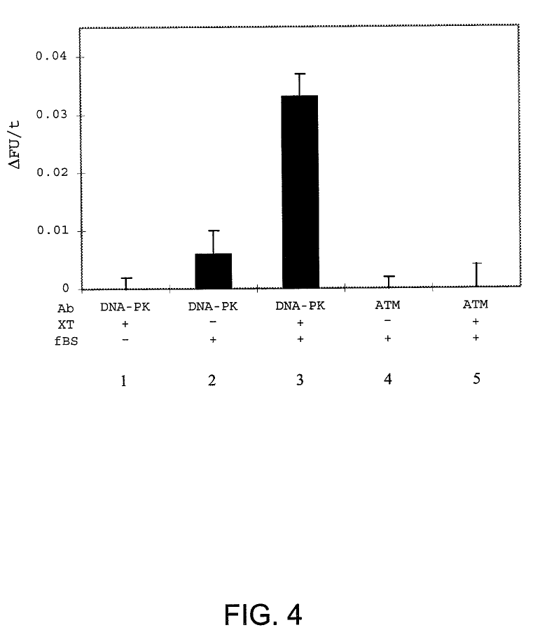

Antibodies to DNA-PK are capable of detecting DNA-PK binding to DNA ends in this assay. Although several methods may be invoked to detect binding of a DSSBP to DNA, including fluorescence, absorbance, or radioactivity, in this example the DNA-PK complex was detected by an anti-DNA-PK antibody (Serotec) which was bound by a secondary antibody conjugated to an enzyme (horseradish peroxidase) that catalyzed a reaction producing fluorescence. FIG. 4 illustrates that the signal is specific to the anti-DNA-PK antibody, since antibodies to ATM, a homolog of DNA-PK, produced no signal in the assay (FIG. 4, compare lanes 3 and 5). Specificity of the interaction is important since this assay proposes the utilization of crude protein extracts to detect a very specific signal. The data of FIG. 3 illustrates the feasibility of this approach by showing that little if any cross-reactivity exists in detecting the DNA-PK complex bound to DNA ends, and that other irrelevant proteins do not produce a signal using this method.

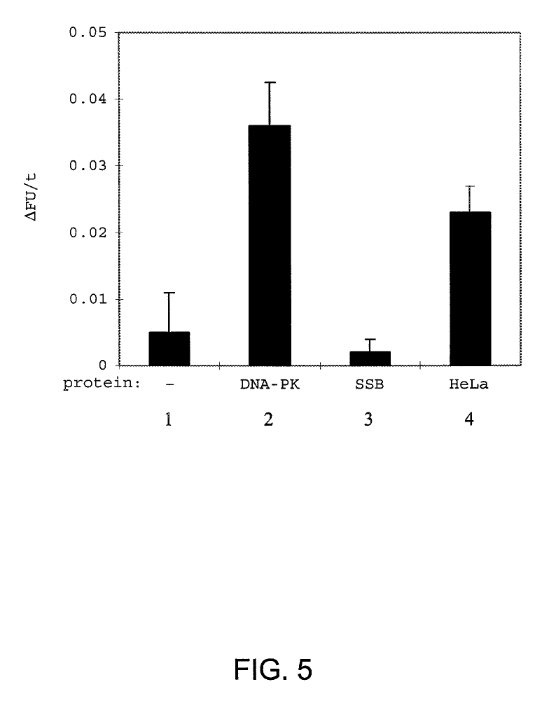

Further proof-of-concept is shown (FIG. 4) by demonstrating that the source of the DSSBP may be pure protein or crude nuclear extract. Purified DNA-PK produces a signal of greater intensity than HeLa nuclear extracts (FIG. 5, compare lanes 2 and 4), whereas the irrelevant protein SSB produces little signal (FIG. 5, lane 3). Thus, this assay is not limited to the utilization of extract as a DSSBP source.

I claim:

1. a method for measuring the DNA-structure specific binding activity of a test protein comprising the steps of:
a) Contacting a nucleic acid substrate comprising a specific structure immobilized on a solid support with a test protein or mixture of proteins
b) detecting the protein or mixture of proteins from step (a) bound to the immobilized nucleic acid substrate.

2. The method according to claim 1, wherein the solid support is a microtiter plate.

3. The method according to claim 1, wherein the immobilized nucleic acid substrate comprises DNA.

4. The method according to claim 3, wherein the DNA is damaged.

5. The method according to claim 1, wherein the nucleic acid structure comprises DNA ends.

6. The method according to claim 4, wherein the damaged DNA comprises UV-irradiated DNA.

7. The method according to claim 1, wherein the test protein comprises a cell extract.

8. The method according to claim 1, wherein the test protein comprises a DNA repair protein.

9. The method according to claim 1, wherein the DSSBP is detected by contacting the solid support of step (b) with an antibody.

10. The method according to claim 9, wherein said antibody comprises an anti-DNA-PK antibody.

11. The method according to claim 8, wherein the DNA repair protein comprises DNA-PK.

12. A method for measuring the DSSBP modulating ability of a test substance comprising the steps of:
a) Contacting a nucleic acid substrate immobilized on a solid support with a test substance to produce a reaction premix
b) Contacting the reaction premix of step (a) with a DSSBP capable of binding the immobilized substrate to produce a reaction mix

13. The method of claim 12, wherein the reaction mix is further subjected to a process whereby the DSSBP is detected.

14. The method according to claim 12, wherein the solid support is a microtiter plate.

15. The method according to claim 12, wherein the immobilized nucleic acid substrate comprises DNA.

16. The method according to claim 15, wherein the DNA is damaged.

17. The method according to claim 12, wherein the nucleic acid structure comprises DNA ends.

18. The method according to claim 16, wherein the damaged DNA comprises UV-irradiated DNA.

19. The method according to claim 12, wherein the DSSBP comprises a DNA repair protein.

20. The method according to claim 13, wherein the DSSBP is detected by contacting the solid support with an antibody.

21. The method according to claim 20, wherein said antibody comprises an anti-DNA-PK antibody.

22. The method according to claim 9, wherein the DNA repair protein comprises DNA-PK.
